(19) United States
(12) Patent Application Publication
(10) Pub. No.: US 2020/0078204 A1
(43) Pub. Date: Mar. 12, 2020

(54) EXTREMITY SUPPORT

(71) Applicant: Purdue Research Foundation, West Lafayette, IN (US)

(72) Inventors: Akshay K. Rao, West Lafayette, IN (US); Tyler Stagge, West Lafayette, IN (US); Hanwen Gu, West Lafayette, IN (US); Sahil Shah, West Lafayette, IN (US)

(73) Assignee: Purdue Research Foundation, West Lafayette, IN (US)

(21) Appl. No.: 16/566,928

(22) Filed: Sep. 11, 2019

Related U.S. Application Data
(60) Provisional application No. 62/730,055, filed on Sep. 12, 2018.

(51) Int. Cl. A61F 5/37; A61F 5/01

(52) U.S. Cl. CPC A61F 5/3761; A61F 5/013; A61F 5/0127; A61F 5/0123

(57) ABSTRACT

An extremity support is disclosed which includes a plate adaptable to be coupled to an extremity fixation apparatus, the plate having a distal end and a proximal end, and a top surface and a bottom surface, wherein the top surface of the plate is adaptable to be coupled to the extremity fixation apparatus, at least one hinge coupled to the bottom surface and about the distal end of the plate, and at least two extendible legs, each articulable about a corresponding hinge from a first angle to a desired angle, each of the two extendible legs configured to provide support for the plate in the vertical direction at the desired angle.

### CROSS-REFERENCE TO RELATED APPLICATIONS

The present patent application is related to and claims the priority benefit of U.S. Provisional Patent Application Ser. No. 62/730,055 filed Sep. 12, 2018, the contents of which are hereby incorporated by reference in its entirety into the present disclosure.

### TECHNICAL FIELD

The present disclosure generally relates to an apparatus for supporting a subject, and in particular relates to providing support for a subject's leg in various situation, e.g., when the subject is sitting down.

### BACKGROUND

This section introduces aspects that may help facilitate a better understanding of the disclosure. Accordingly, these statements are to be read in this light and are not to be understood as admissions about what is or is not prior art.

Leg braces are commonplace. They are utilized for individuals with limited mobility or for those who need to mobilize their extremities in a preselected angle for a long duration of time, e.g., post-surgery. For example, for a period of time after a knee surgery the patient is asked to keep his/her leg in a straight position to allow for healing to take place. Similarly, when a person has fractured their leg, e.g., a tibial fracture, they are asked to maintain their leg in a cast in a straight position to allow healing to occur.

Users face substantial difficulty due to the typical high frequency and duration of sitting. Difficulty is most commonly caused because most leg braces extend up to the hip level, thus interfering with the contact between a person and his or her chair. As a result, the user is usually forced into an uncomfortable and difficult-to-maintain position. A commonly used solution is to either sit on the edge of the chair or use another chair to support the foot of the injured leg. These solutions can cause discomfort over extended periods of time and are not accessible in every environment. In addition, propping the leg on another chair can cause the knee to tend to acquire a negative angle thereby causing pain and discomfort.

Therefore, there is an unmet need for a novel approach for a leg brace or cast to be supported in a way so as to reduce discomfort.

Sitting upright in a variety of situations can be uncomfortable. Variations in seat height, seat length, chair height, and angles relative to the user can influence the comfort level that the user experiences. A commonly used solution to supplement comfort is by elevating the legs to the hip level. This is commonly experienced by propping feet up on an adjacent chair or ledge. However, with rapidly changing environments that one experiences throughout a day, there doesn't exist a compact, mobile solution to elevate the leg agnostic of the environment.

### SUMMARY

An extremity support is disclosed. The extremity support includes a plate that is adaptable to be coupled to an extremity fixation apparatus. The plate has a distal end and a proximal end, and a top surface and a bottom surface. The top surface of the plate is adaptable to be coupled to the extremity fixation apparatus. The extremity support also includes at least one hinge that is coupled to the bottom surface and are positioned about the distal end of the plate. In addition, the extremity support includes at least two extendible legs. Each leg is articulable about a corresponding hinge from a first angle to a desired angle. Each of the two extendible legs are configured to provide support for the plate in the vertical direction at the desired angle.

A method of providing support for an extremity fixation apparatus is disclosed. The method includes the step of providing an extremity support having a plate, wherein the plate has a top surface, a bottom surface, a proximal end, a distal end. The extremity support further comprises at least one hinge coupled to the bottom surface about the distal end of the plate, and at least two extendible legs configured to articulate about a corresponding hinge of the at least one hinge from a first angle to a desired angle. The method also includes coupling the extremity support to said extremity fixation apparatus. The method further includes adjusting the extremity support to fit a user's extremity. Additionally, the method also includes deploying the at least two extendible legs from the first angle to the desired angle such that the at least two extendible legs support the user's extremity.

### BRIEF DESCRIPTION OF DRAWINGS

FIG. 1 is a depiction of the extremity support system, according to the present disclosure which shows a perspective view of the extremity support with two legs extended downward.

FIG. 2 is a sideview of an embodiment of the extremity support system of FIG. 1.

FIG. 3 is a front view of an embodiment of the extremity support system of FIG. 1.

FIG. 4 is a bottom view of an embodiment of the extremity support of FIG. 1.

FIG. 5A is a closeup of an embodiment of the extremity support system of FIG. 1 with the legs articulated into an extended position.

FIG. 5B is a closeup of an embodiment of the extremity support system of FIG. 1 with the legs articulated into a folded position.

FIG. 6 is a flowchart of how the extremity support system of FIG. 1 can be used by a user.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

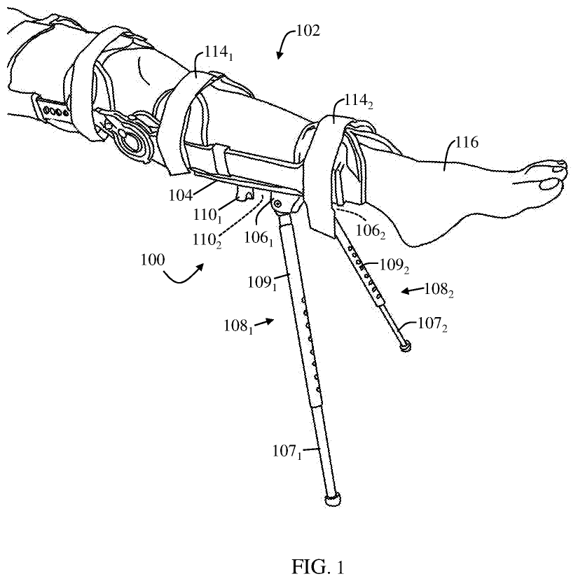

A novel approach for a brace 102 or cast positioned on an extremity to be supported in a way so as to reduce discomfort and also provide therapy is disclosed. In the present disclosure, the phrase extremity 116 (shown in FIG. 1) refers to any part of the legs and arms of an individual. An embodiment of a proposed extremity support 100 is shown in FIGS. 1, 2, 3, 4, 5A, and 5B in various angles and in operation with a brace 102. However, the same can be adapted for a leg cast. The extremity support 100 includes a support plate 104 two hinges 106₁ and 106₂ mounted on a distal end of the support plate 104, and two legs 108₁ and 108₂ extending from the support plate 104 and configured to be articulating about the respective hinges 106₁ and 106₂ from a folded position to a deployed position forming a maximum angular relationship of about 120° from the folded position. The support system further includes two folding members 110₁ and 110₂ adapted to receive the respective legs 108₁ and 108₂. In the folded position, the legs 108₁ and 108₂ have to be snapped out of the respective folding members 110₁ and 110₂ in order to articulate about the respective hinges 106₁ and 106₂ from the folded position to the maximum angular position of about 120°.

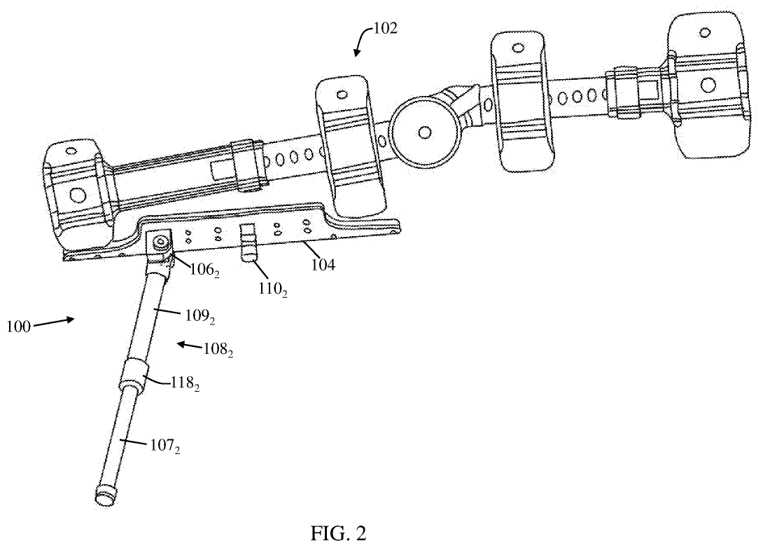

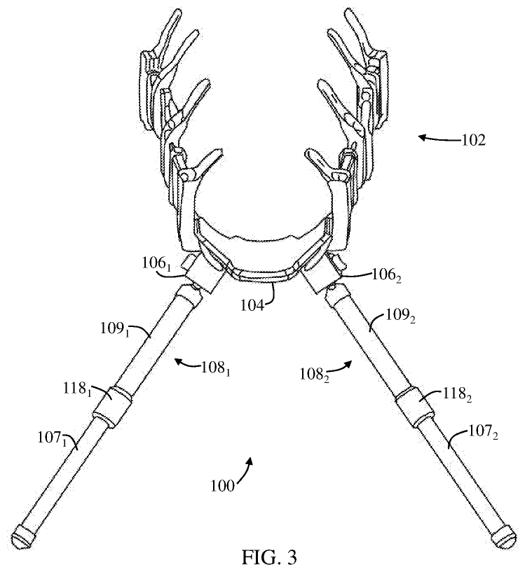

The legs 108₁ and 108₂ of the extremity support 100 are extendible, and can be extended into a first position and remain in that position until the legs 108₁ and 108₂ are extended into a second position. The legs 108₁ and 108₂ are extendible by utilizing a telescopic approach. Different approaches can be used to move a first portion 107₁ and 107₂ of a respective leg 108₁ and 108₂ slidably within a second portion 109₁ and 109₂ of the respective leg 108₁ and 108₂ from the first position to the second position. For example, the first portion 107₁ and 107₂, which has a smaller diameter than the corresponding second portion 109₁ and 109₂, may include spring-loaded dimples matching holes disposed on the second portion 109₁ and 109₂ (shown in FIG. 1). The first portion 107₁ and 107₂ can be moved with respect to the second portion 109₁ and 109₂, by pressing on the dimple until the dimple is inside the corresponding second portion 109₁ and 109₂ and then forcibly moving the corresponding first portion 107₁ and 107₂ with respect to the corresponding second portion 109₁ and 109₂. Alternatively, a collet 118₁ and 118₂ (shown in FIG. 2 & FIG. 3) can be used to extend or retract the legs 108₁ and 108₂. By loosening the respective collet's 118₁ and 118₂ nut, the corresponding first portion 107₁ and 107₂ can be moved within the corresponding second portion 109₁ and 109₂ and upon reaching the desired position, the nut can then be retightened to lock the placement of the first portion 107₁ and 107₂ with respect to the corresponding second portion 109₁ and 109₂. Other interfaces, known to a person having ordinary skill in the art are also possible.

The hinges 106₁ and 106₂ of the extremity support 100 are designed to allow articulation of the corresponding legs 108₁ and 108₂ up to a limit. For example, the angular relationship of the leg 108₁ and 108₂ from a folded position, where a leg 108₁ or 108₂ is folded against the support plate 104, to a full articulated position may result in a total of 120° at which point the legs 108₁ and 108₂ reach a hard stop at the corresponding hinges 106₁ and 106₂.

Referring to FIG. 1, a perspective view of an embodiment of the extremity support 100 in use. The extremity support 100 is shown attached to a brace 102 which is attached to an extremity 116 of a user. The brace 102 shown in this embodiment is a leg brace and, respectively, the extremity 116 shown is a leg. In the embodiment shown, the extremity support 100 is attached to the brace 102 through the use of straps 114₁ and 114₂, which connect the upper surface of the support plate 104 to the brace 102. The two legs 108₁ and 108₂ of the extremity support 100 are extended downward and the user's extremity 116 is extended outward horizontally indicating that the user is currently in a sitting position and that the extremity support 100 is currently supporting the brace 102 and extremity 116. The two legs 108₁ and 108₂ are each attached to a corresponding hinge 106₁ and 106₂, wherein the hinges 106₁ and 106₂ attached about a distal end on the bottom surface of the support plate 104. The legs 108₁ and 108₂ in this embodiment are extendible through spring-loaded dimples on the corresponding first portion 107₁ and 107₂ matching holes on the corresponding second portion 109₁ and 109₂. In this embodiment the extremity support 100 further includes folding members 110₁ and 110₂ adapted to receive the corresponding legs 108₁ and 108₂.

Referring to FIG. 2 and FIG. 3, a side view (FIG. 2) and front view (FIG. 3) of an embodiment of the extremity support 100 and brace 102 shown in FIG. 1 is presented. The embodiment shown here is similar to FIG. 1 in that the extremity support 100 comprises a support plate 104, having two hinges 106₁ and 106₂ attached about the distal end on the bottom surface of the support plate 104. Also, the extendable legs 108₁ and 108₂ is attached to the corresponding hinge 106₁ and 106₂ and is depicted in an extended position. The extendable leg 108₁ and 108₂ has a corresponding first portion 107₁ and 107₂ and a corresponding second portion 109₁ and 109₂, wherein said first portion 107₁ and 107₂ has a smaller diameter than said corresponding second portion 109₁ and 109₂ and each first portion 107₁ and 107₂ is configured to move slidably within the corresponding second portion 109₁ and 109₂. The embodiment shown here differs from FIG. 1 in that the extendable legs 108₁ and 108₂ depicted are extendable through the use of a collet 118₁ and 118₂ rather than through the use of the spring-loaded dimple and hole embodiment shown in FIG. 1.

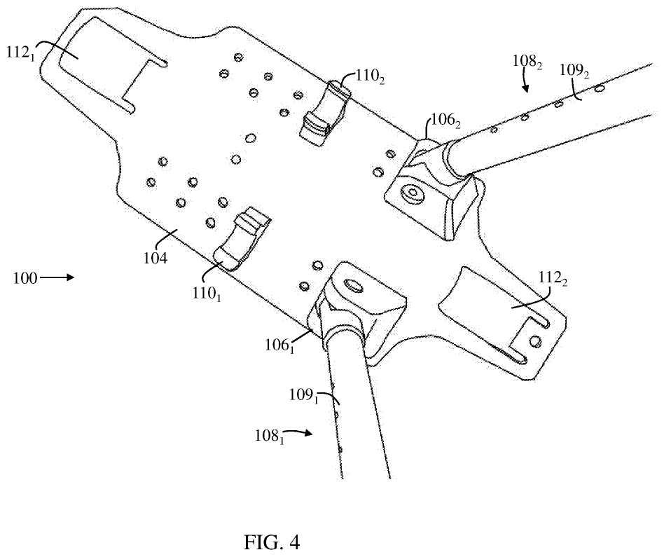

Referring to FIG. 4, a bottom view of an embodiment of the extremity support 100 and brace 102 shown in FIG. 1 is presented. The support plate 104 is shown with the bottom surface of the support plate 104 face up. Attached to the bottom surface about the distal end of the support plate 104 are two hinges 106₁ and 106₂ wherein, each hinge 106₁ and 106₂ has a corresponding leg 108₁ and 108₂ attached to it. The legs 108₁ and 108₂ depicted in FIG. 4 are extendable through the use of a spring-loaded dimple, on each of the said first portions 107₁ and 107₂ (shown in FIG. 1), and a series of holes on each of the said second portions 109₁ and 109₂ as described above. There are also two folding members 110₁ and 110₂ configured to receive said legs 108₁ and 108₂ such that when the legs 108₁ and 108₂ are in the folded position they must be snapped out of the respective folding member 110₁ and 110₂ in order to articulate about the corresponding hinge 106₁ and 106₂. With further reference to FIG. 4, there are two attachment slots 1121 and 1122 at the proximal and distal end of the support plate 104, respectively. These attachment slots 1121 and 1122 are each adapted to receive an attachment member that can attach the extremity support 100 to a brace 102 (shown in FIG. 1) or a cast.

Typical material for the components of the extremity support 100 may include metal (e.g., steel, aluminum, alloys, etc.) or plastic for the support plate 104 and metal, or plastic for the legs 108₁ and 108₂.

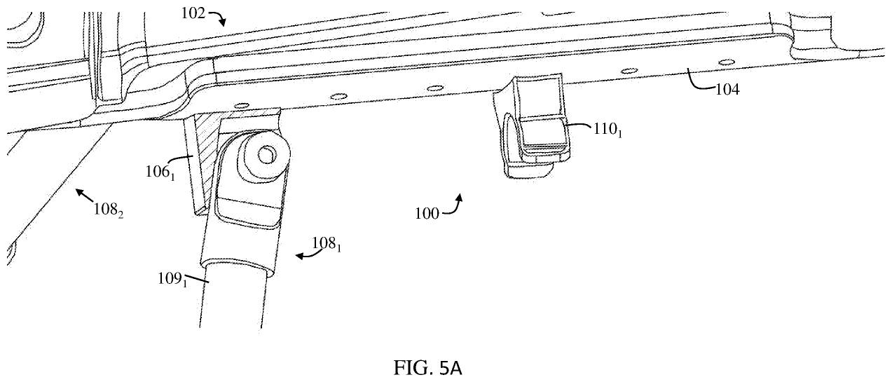

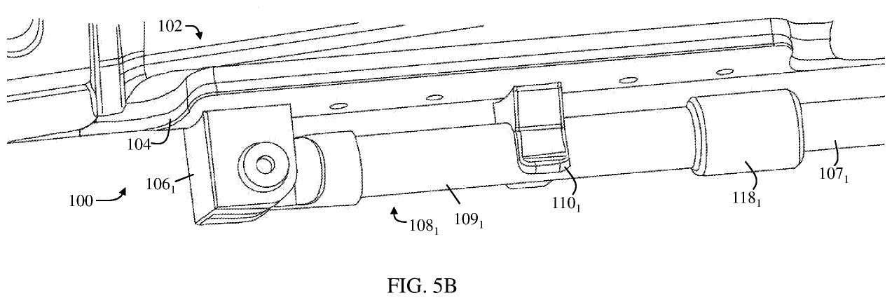

Referring to FIG. 5A and FIG. 5B, a zoomed side view of the extremity support 100 of FIG. 1 and brace 102 is presented depicting the leg 108₁ in a deployed position, in FIG. 5A, and a folded position, in FIG. 5B. In FIG. 5A, the leg 108₁ is articulated about the corresponding hinge 106₁ from the folded position to a fully deployed position of about 120° from the folded position. FIG. 5B depicts the leg 108₁ in a folded position where the second portion 109₁ of the leg 108₁ is snapped in to the folding member 110₁.

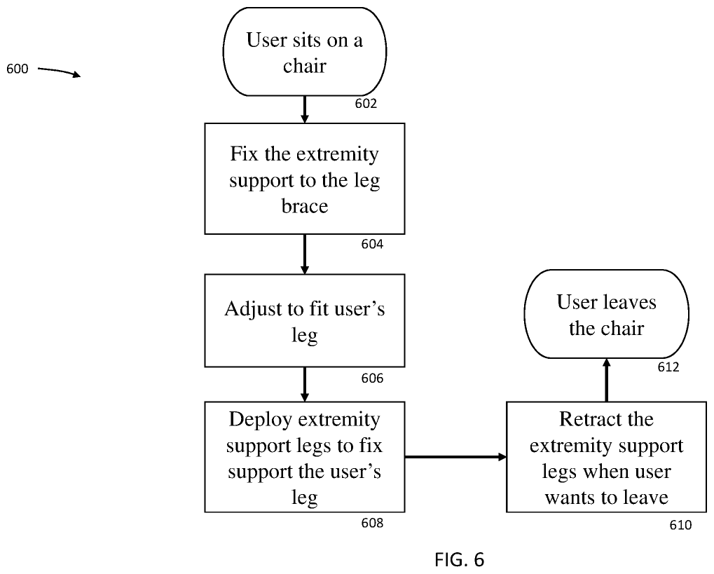

Referring to FIG. 6, a flowchart is presented on a process 600 of how to use the extremity support system 100. The flowchart 600 begins by the user sitting on a chair or other seating arrangements (block 602). Next, the user fixes the extremity support 100 to the leg brace (block 604), which can be done through the use of belts. Next, the user adjusts the fit of the extremity support 100 to the user's leg (block 606). Next, the users deploys the legs 108₁ and 108₂ of the extremity support 100 (shown in FIG. 1) from the folded position to the deployed position (block 608), forming a bipod. The next two blocks allow for the user to retract the bipod (block 610) into the folded position and leave the chair (block 612).

According to one embodiment of the support system, the legs 108₁ and 108₂ (shown in FIG. 1) are extendible and retractable by an electromechanical subsystem utilizing linear actuators (e.g., a stepper motor and rack and pinion), known to a person having ordinary skill in the art. This electromechanical subsystem is mounted between the support plate 104 and the legs 108₁ and 108₂. A controller and supporting circuitry can process signals from a positional sensor (e.g., a digital level, e.g., mounted on the support plate) and activate the electromechanical devices. The positional sensor provides signals to the controller to control the extension of the legs 108₁ and 108₂ and thus the angle of the support plate 104. This embodiment allows the extremity support 100 (shown in FIG. 1) to not only be used as a system to support an extremity in a comfortable position, but also provide therapy of cyclic motion for post-operative rehabilitation as well as provide other therapeutic benefits.

Those having ordinary skill in the art will recognize that numerous modifications can be made to the specific implementations described above. The implementations should not be limited to the particular limitations described. Other implementations may be possible.

1. An extremity support, comprising:
a plate adaptable to be coupled to an extremity fixation apparatus, the plate having a distal end and a proximal end, and a top surface and a bottom surface, wherein the top surface of the plate is adaptable to be coupled to the extremity fixation apparatus;
at least one hinge coupled to the bottom surface and about the distal end of the plate; and
at least two extendible legs, each articulable about a corresponding hinge from a first angle to a desired angle;
each of the two extendible legs configured to provide support for the plate in the vertical direction at the desired angle.

2. The extremity support of claim 1, wherein the plate is coupled to the extremity fixation device by one or more straps.

3. The extremity support of claim 1, wherein the extremity fixation device can be any one of: a leg brace, a leg cast, an arm brace, or an arm cast.

4. The extremity support of claim 1, wherein the plate is made from one or more of metal, and plastic.

5. The extremity support of claim 1, wherein the at least two extendible legs are made from one or more of metal, and plastic.

6. The extremity support of claim 1, further comprising at least two folding members coupled to the bottom side of the plate and each configured to receive a corresponding of the at least two extendible legs in the first angle.

7. The extremity support of claim 1, wherein the desired angle is 120° or less than the first angle.

8. The extremity support of claim 1, further comprising an electromechanical subsystem adaptable to cycle the extendible legs from a first height to a second height translating position of the support plate from a first cyclic angle to a second cyclic angle.

9. The extremity support of claim 8, the electromechanical subsystem includes a stepper motor and a rack and pinion assembly.

10. The extremity support of claim 9, further comprising a positional sensor providing angular position of the plate.

11. The extremity support of claim 10, the positional sensor is a digital level.

12. A method of providing support for an extremity fixation apparatus, comprising:
providing an extremity support having a plate, wherein the plate has a top surface, a bottom surface, a proximal end, a distal end, wherein the extremity support further comprises at least one hinge coupled to the bottom surface about the distal end of the plate, and at least two extendible legs configured to articulate about a corresponding hinge of the at least one hinge from a first angle to a desired angle;
coupling the extremity support to said extremity fixation apparatus;
adjusting the extremity support to fit a user's extremity; and
deploying the at least two extendible legs from the first angle to the desired angle such that the at least two extendible legs support the user's extremity.

13. The method of claim 12, wherein the plate is coupled to the extremity fixation device by one or more straps.

14. The method of claim 12, wherein the extremity fixation device can be any one of: a leg brace, a leg cast, an arm brace, or an arm cast.

15. The method of claim 12, wherein the plate is made from one or more of metal, and plastic.

16. The method of claim 12, wherein the at least two extendible legs are made from one or more of metal, and plastic.

17. The method of claim 12, further comprising:
providing at least two folding members coupled to the bottom side of the plate and each configured to receive a corresponding of the at least two extendible legs in the first angle.

18. The method of claim 12, further comprising:
providing an electromechanical subsystem adaptable to cycle the extendible legs from a first height to a second height translating position of the support plate from a first cyclic angle to a second cyclic angle, wherein the electromechanical subsystem includes a stepper motor and a rack and pinion assembly.

19. The method of claim 18, further comprising:
providing a positional sensor providing angular position of the plate.

20. The method of claim 19, wherein the positional sensor is a digital level.
